(19) United States
(12) Patent Application Publication
(10) Pub. No.: US 2009/0104656 A1
(43) Pub. Date: Apr. 23, 2009

(54) System for the Inducible Expression of Recombinant Proteins in Cyanobacteria

(75) Inventors: Etienne Weiss, Fegersheim (FR); Dominique Desplancq, Strasbourg (FR)

Correspondence Address: FULBRIGHT & JAWORSKI L.L.P., 600 CONGRESS AVE., SUITE 2400, AUSTIN, TX, 78701, US

(73) Assignee: Universite Louis Pasteur, Strasbourg (FR)

(21) Appl. No.: 10/596,002

(22) PCT Filed: Nov. 22, 2004
(86) PCT No.: PCT/FR04/02976

§ 371 Date: Dec. 10, 2008

(30) Foreign Application Priority Data
Nov. 24, 2003 (FR) 0313736

(51) Int. Cl. C12P 21/00; C12N 15/63; C12N 1/21

(52) U.S. Cl. 435/69.1; 435/320.1; 435/252.3

(57) ABSTRACT

The invention relates to a system for inducible expression in cyanobacteria, which enables the expression of recombinant proteins, and to the vectors and cyanobacteria containing said expression system.

The present invention relates to a system for inducible expression in cyanobacteria, which allows the expression of recombinant proteins, and also to the vectors and cyanobacteria containing this expression system.

Recent developments in nuclear magnetic resonance (NMR) now make it possible to study large biomolecules such as proteins. However, a uniform minimum enrichment in ¹³C and ¹⁵N of 90% is required in order to allow an NMR analysis. An additional enrichment in ²H can also prove to be necessary for proteins of molecular weight greater than 20 kDa.

Up until now, the labeling process consisted in producing the protein of interest mainly in E. coli and in culturing this microorganism on labeled media, i.e. media enriched in a stable isotope. However, even though these media are available on the market, they are extremely expensive, particularly as regards ¹³C labeling and ²H labeling. The production of labeled proteins for NMR therefore proves to be a difficult and expensive process.

Cyanobacteria are photoautotrophic organisms. These bacteria are capable of growing on a minimum medium containing carbonate as sole carbon source, nitrites, nitrates or ammonium as sole nitrogen source and mineral salts. Energy is provided by light via photosynthesis.

Patent application FR 2 820 758 describes a system for the expression and constitutive labeling of recombinant proteins in the cyanobacterium Anabaena using the tac promoter of the E. coli bacterium. The system developed in that patent application thus makes it possible to use, for the growth of the cyanobacterium, a relatively inexpensive labeled medium containing Na¹⁵NO₃ and NaH¹³CO₃ as labeled nitrogen and carbon sources.

However, although it is easy to produce nontoxic proteins such as the last 24 N-terminal kDa of the gyrase B domain of E. coli or the maltose-binding protein (MBP), the expression of certain eukaryotic proteins proves to be much more difficult in cyanobacteria using a constitutive promoter. The expression of these proteins is in fact lost because of a problem of plasmid rearrangement during bacterial growth. The development of a system for efficient expression in cyanobacteria is consequently an important problem with regard to the labeling of recombinant proteins.

The applicant has discovered that it is possible to express potentially toxic proteins under the control of an inducible promoter in cyanobacteria.

A subject of the invention is thus a method for expressing recombinant proteins in cyanobacteria using an inducible cyanobacterial transcription promoter sequence.

Another subject of the invention consists of a vector containing a sequence encoding a protein under the control of an inducible cyanobacterial transcription promoter sequence.

Another subject of the invention consists of the use of a vector of this type in cyanobacteria for expressing recombinant proteins in these bacteria.

Another subject of the invention consists of a cyanobacterium containing a vector of this type.

Other subjects of the invention will become apparent in the light of the description, of the examples which follow and of the drawings attached to the present application.

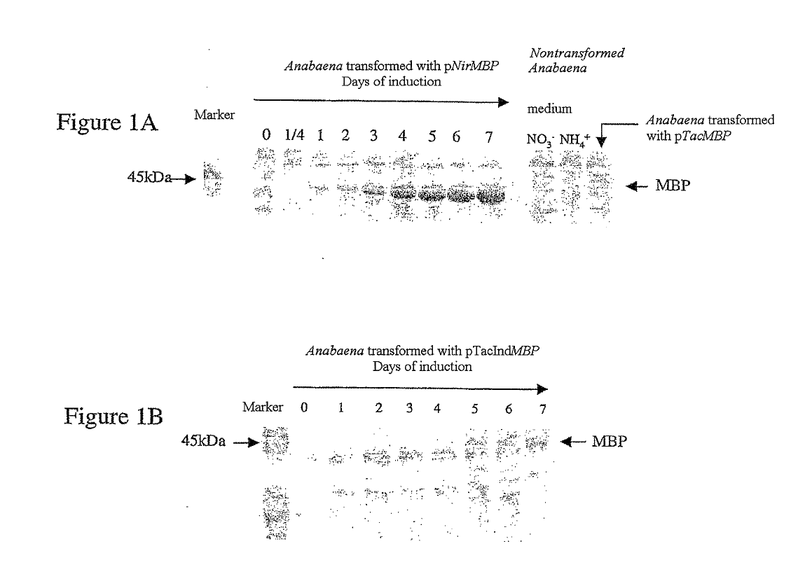

FIG. 1A shows the accumulation of the MBP protein as a function of time (in days) in an Anabaena sp. PCC 7120 bacterium transformed with the plasmid pNirMBP and induced.

FIG. 1B shows the accumulation of the MBP protein as a function of time (in days) in an Anabaena sp. PCC 7120 bacterium transformed with the plasmid pTacIndMBP and induced.

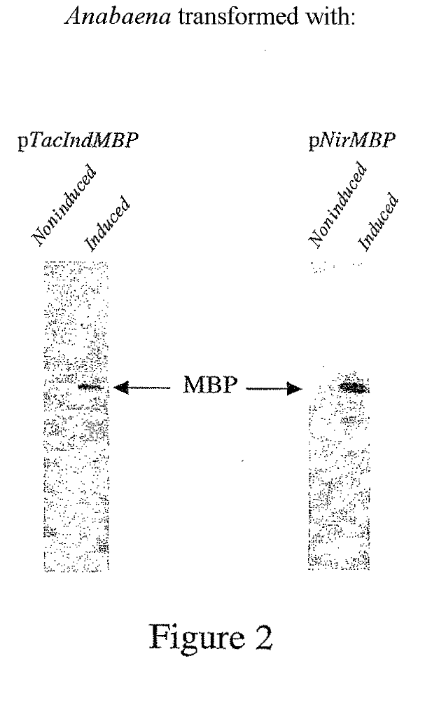

FIG. 2 shows a comparison of the amounts of proteins synthesized before and after induction of Anabaena sp. PCC 7120 bacterium transformed either with the plasmid pNirMBP or with the plasmid pTacIndMBP.

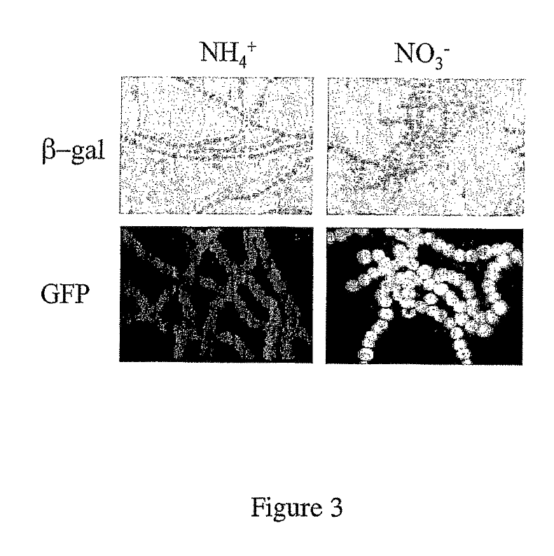

FIG. 3 shows Anabaena cells expressing respectively β-galactosidase (photograph with a visible light microscope) and GFP (green fluorescent protein) (photograph with a fluorescence microscope) under inducing conditions (NO₃⁻) or not expressing these proteins in the presence of the NH₄⁺ repressor.

The invention thus relates to a method for expressing recombinant proteins, characterized in that it consists in introducing into cyanobacteria a sequence encoding a protein downstream of an inducible cyanobacterial transcription promoter sequence, and then in inducing the expression of this protein and isolating the recombinant proteins thus expressed.

Unlike E. coli, no commercially available expression system exists in cyanobacteria. Only a few examples of heterologous expressions in cyanobacteria have been reported in the literature, and the expression of these proteins is generally under the control of a constitutive cyanobacterial promoter. Inducible expression in cyanobacteria could be carried out through the use of inducible promoters that have been found to function in cyanobacteria, such as the lambda bacteriophage P_{L} promoter (λP_{L}), the E. coli trc promoter or the E. coli tac promoter. These three promoters require the expression of their own repressor in the cyanobacterium in order to allow regulation of the promoter. The λP_{L} promoter which is inducible by means of an increase in temperature to 42° C., is not suitable in our case, since Anabaena sp. PCC 7120 cannot grow at temperatures above 30° C. The tac and trc promoters are two promoters which function similarly; they are both repressed by the LacI repressor of the lac operon and induced, in the presence of IPTG, a lactose analog. However, expression trials with the tac promoter have proved to be disappointing. The amount of recombinant protein expressed after induction is very low (approximately 10 times less than that obtained with a constitutive tac promoter (expression in the absence of the LacI repressor in the cell)) and never reaches the protein levels that result from constitutive expression as described in the publications. Thus, the tac promoter does not appear to be an appropriate inducible promoter. Furthermore, cyanobacteria do not possess the lacy lactose permease gene which allows efficient entry of the IPTG inducer into the cells.

To date, very few inducible promoters have been characterized in cyanobacteria. Certain cyanobacterial promoters are inducible with metals or minerals. Their use is, however, limited since they are induced at very low concentrations of inducer of the order of one micromolar, and the culture medium generally used for the growth of cyanobacteria already contains sufficient amounts of metals required for the induction of these promoters.

One of the promoters most well characterized in cyanobacteria is the promoter which controls the expression of the nir operon, which is involved in nitrate assimilation in cyanobacteria. This operon is induced in the presence of nitrate and is also well repressed by ammonium (Frias, J. E. et al., 1997, J. Bacteriol., 179, 477-486).

In fact, in cyanobacteria, the genes involved in nitrate assimilation are encoded in the nir operon, the transcription of which is controlled by the nir promoter. The activation of this promoter requires the binding to the DNA of two transcription regulators, NtcA and NtcB, necessary for the expression of the genes encoding proteins specifically involved in nitrate assimilation (Frias, J. E. et al., 1997, J. Bacteriol., 179, 477-486).

Thus, the method for expressing recombinant proteins according to the invention uses, as transcription promoter sequence, that of the cyanobacterial nir operon, which sequence is induced in the presence of nitrate and/or of nitrite in the medium, and preferably in the presence of NaNO₃.

It is important to note that the initiation of the nir promoter in cyanobacteria is extremely rapid and is induced a few hours after incubation of the bacteria in a medium containing nitrate as nitrogen source in place of ammonium.

The cyanobacterium used is preferably of the species Anabaena, and more particularly Anabaena sp. PCC 7120 (strain available at the Culture Collection of the Institut Pasteur, Paris), a filamentous bacterium which is capable of growing on minimum media containing ¹³C and/or ¹⁵N and/or ²H so as to produce, at low cost, labeled recombinant proteins for NMR analysis.

Preferably, the culture medium for these cyanobacteria that is used for the labeling of expressed recombinant proteins contains at least Na¹⁵NO₃.

In the constitutive expression system (the protein expression takes place throughout the cell cycle) used, it is impossible to produce eukaryotic proteins which were toxic to the cell. The constitutive expression of such proteins in the cell is reflected by a loss of the expression due to a rearrangement of the expression vector.

The invention thus relates to the expression of recombinant proteins, in which the recombinant protein expressed is toxic for the cyanobacteria.

In fact, this nitrate-inducible expression system based on the promoter of the cyanobacterial nir operon makes it possible to control the expression of the protein of interest. In the absence of inducer in the culture medium, the protein is not produced since the promoter is repressed. The induction of the expression is obtained when the nitrogen source in the medium is either nitrates or nitrites or both.

Another subject of the invention is also a vector containing a DNA sequence encoding a recombinant protein under the control of an inducible cyanobacterial transcription promoter sequence.

The DNA sequence comprising the genetic information required for the expression of a recombinant protein under the control of a transcription promoter sequence according to the invention can be included in any vector commonly used by those skilled in the art.

Preferably, the inducible cyanobacterial transcription promoter sequence is that of the cyanobacterial nir operon.

The present invention also relates to the use of a vector according to the invention, for expressing recombinant proteins in cyanobacteria.

Preferably, the vectors are used in cyanobacteria cultured in a medium which contains ¹³C and/or ¹⁵N and/or ²H, and even more particularly in a medium which contains at least Na¹⁵NO₃.

Another subject of the invention is also a cyanobacterium transformed with a vector according to the invention. Preferably, this cyanobacterium is of the species Anabaena, and more preferably Anabaena sp. PCC 7120.

This expression system makes it possible to solve the problems of loss of expression encountered during the production of toxic proteins in a constitutive system. The expression levels obtained using this system are very high, of the order of 100 mg/l. This system shows effectiveness equivalent to the best expression systems developed in E. coli, in particular the T7 system (Studier et al., 1986, J. Mol. Biol. 189, 113-130). The recombinant protein can in fact represent more than 30% of the total cell proteins.

This system is therefore particularly suitable for the production of milligram amounts of ¹³C, ¹⁵N and ²N labeled proteins for NMR. The labeled substrates are added only at the moment the expression of the protein is induced, which makes it possible to decrease the labeling costs compared with the constitutive expression system. In fact, with the constitutive tac system (Desplancq et al., 2001, Protein Express. Purif. 23, 201-217), during ¹³C labeling in a fermenter, 90% of the NaH¹⁵CO₃ is lost in the form of CO₂ during the aeration of the fermenter with argon and must be compensated for through regular additions of NaH¹³CO₃ throughout the duration of the fermentation (8 to 10 days).

In the system according to the invention, the use of NaH¹³CO₃ only during the period of expression (5 days) of the recombinant protein thus limits the amounts of labeled substrate NaH¹³CO₃ used and therefore decreases the costs of the ¹³C labeling.

Thus, these bacteria are capable of overproducing labeled recombinant proteins with a degree of isotopic enrichment equivalent to that obtained in E. coli, but at a cost which is approximately 10 times lower.

The high expression levels in the inducible expression system according to the invention make it possible not only to decrease the culture volumes used and, consequently, the labeling costs, but also to more readily produce deuterated proteins since, in E. coli, culturing in deuterated medium generally leads to a 3- to 4-fold decrease in expression level. Furthermore, the cyanobacteria use ²H₂O directly as deuterated substrate. This makes it possible to recycle the deuterated medium and to reuse it for further labelings. The ²H-labeling of recombinant proteins in cyanobacteria therefore makes it possible to significantly decrease the cost of ²H-labeling.

The following examples illustrate the invention without in any way limiting it.

### EXAMPLE NO. 1

### Construction of the Vector pTac and of its Derivatives pTacMBP, pTacGyrB(1-219), pTacGST-E6, ptacMBP-E6 and ptacMBP-YZD2

The vector pRL25Cmcs was obtained by digesting the vector pRL25C (Wolk et al., 1988, J. Bacteriol. 170, 1239-1244) with the NotI and BamHI restriction enzymes according to the producer's instructions (New England Biolabs, Beverly, Mass., USA) so as to introduce therein a cloning site for the StuI, XhoI and SmaI restriction enzymes, using the following oligonucleotide primers: 5′-GGCCGCAGGCCTCTCGAGCCCGGGG and 5′-GATCCCCCGGGCTCGAGAGGCCTGC. The fragment encoding the tac promoter was obtained by digestion of the vector pKK223.3 (Amersham Biosciences, Uppsala, Sweden) with the XmnI and SspI restriction enzymes (New England Biolabs, Beverly, Mass., USA). This fragment was then ligated into the vector pRL25Cmcs digested beforehand with SmaI and dephosphorylated with calf intestine phosphatase (New England Biolabs) to give the vector pTac. The genes encoding the GST-E6 protein (fusion protein comprising glutathione-5-transferase (GST) and the E6 protein of the HPV16 virus), the MBP-E6 protein (fusion protein comprising the maltose-binding protein (MBP) and the E6 protein of the HPV16 virus) and the MBP-YZD2 protein (fusion protein comprising the maltose-binding protein (MBP) and the C-terminal domain of the E6 oncoprotein (YZD2)) were obtained by PCR from the plasmids pETGST-E6, pETMBP-E6 and pETMBP-YZD2. The latter vector is identical to the vector MBP-E6-C4C/4S (Nominé et al., 2003 Biochem. 42, 4909-4917). The vectors pETGST-E6 and pETMBP-E6 were constructed as follows: the gene encoding the E6 protein was obtained by PCR with the following oligonucleotides: 5′-ATCCGGGGTCTCCCATGTTTCAGGACCCACAGGAGCGAC and 5′-ATCCGGGGTCTCGGTACCGCGGCCGCTTACAGCTGGGTTTCTCTACGTGTTC, using, as template, the vector MBP-E6 6C/6S (Nominé et al., 2001, Protein Eng. 14, 297-305). This PCR fragment was then digested with the NcoI and KpnI restriction enzymes and ligated with the vector pETM-30 linearized beforehand with the NcoI and KpnI enzymes, to give the vector pETGST-E6. This same PCR fragment encoding the E6 protein and digested with the NcoI and KpnI restriction enzymes was also ligated with the vector pETM-41 linearized beforehand with the NcoI and KpnI enzymes, to give the vector pETMBP-E6. The vectors pETM-30 and pETM-41 were obtained from Dr. Stier and are referenced on the site www.embl-heidelberg.de/Externalinfo/geerlof/draft frame/index.html.

The PCR fragments encoding the GST-E6, MBP-E6 and MBP-YZD2 proteins originating, respectively, from the plasmids pETGST-E6, pETMBP-E6 and pETMBP-YZD2 were then cloned between the EcoRI and BamHI restriction sites located in the multiple cloning site of the vector pTac, so as to generate the vectors pTacGST-E6, ptacMBP-E6 and pTacMBP-YZD2. The fragment encoding the tac promoter and the gene for MBP was obtained by digesting the vector pMALc2 (New England Biolabs, Beverly, Mass., USA) with the SspI and BamHI restriction enzymes. This fragment was then ligated into the vector pRL25C linearized as follows, to give the vector pTacMBP. pRL25C was linearized with the NotI restriction enzyme, then treated with the Klenow enzyme, redigested with the BamHI restriction enzyme and, finally, dephosphorylated with calf intestine phosphatase (New England Biolabs). The vector pTacGyrB(1-219), which contains the N-terminal domain of E. coli gyrase B under the control of the constitutive tac promoter, corresponds to the vector pRL25C24K (Desplancq et al., 2001, Protein Express. Purif. 23, 201-217).

### EXAMPLE NO. 2

### Construction of the Expression Vector pNir and of its Derivatives pNirMBP, pNirGyrB(1-219), pNirMBP-YZD2, pNirMBP-E6, pNirGFP, pNirLacZ and pNirGST-E6

The expression vector pNir was constructed from the vector pRL25Cmcs. The fragment encoding the Nir promoter was obtained by polymerase chain amplification (PCR) using the vector pCSE21 (Frias et al., 1997, J. Bacteriol. 179, 477-486) as template and the oligonucleotides: 5′-GCGCGCAGATCTAGCTACTCATTAGTTAAGTGTAATG and 5′-GGCCGGGGATCCGAATTCGTTCTCATAAAGTTTTTTTGCTCAAG. This fragment was then digested with the BglII and BamHI restriction enzymes, then ligated into the vector pRL25Cmcs digested beforehand with BamHI and dephosphorylated with calf intestine phosphatase (New England Biolabs), to give the vector pNir. The nir promoter can be cloned in both orientations into the vector pRL25Cmcs. For all the expression experiments, the orientation which generates, downstream of the promoter, a multiple cloning site containing EcoRI, BamHI, StuI, XhoI, SmaI and NotI was used. All the coding sequences of the recombinant proteins tested (MBP, GyrB(1-219), GST-E6, MBP-E6, GFPuv (green fluorescent protein, Crameri et al., (1996) Nature Biotechnol., 14: pp 315-19, β-galactosidase of E. coli), MBP-YZD2), in this vector, were cloned using the EcoRI and BamHI restriction sites specially introduced for this purpose downstream of the nir promoter.

### EXAMPLE NO. 3

### Construction of the Vector pTacIndMBP

The vector pRL25C was linearized with the NotI restriction enzyme, and then treated with the Klenow enzyme. The vector was then redigested with the BamHI restriction enzyme and dephosphorylated with calf intestine phosphatase (New England Biolabs). The MscI/BamHI fragment of the vector pMALc2 was ligated into the linearized vector pRL25C, to give the vector pTacIndMBP.

### EXAMPLE NO. 4

### Transformation of the Cyanobacterium and Amplification of the Transformants

All the cultures of Anabaena sp. PCC 7120 on solid and liquid medium were realized at a temperature of 28° C. with an illumination of 1500 lux. The culture medium used is BG-11 medium (Castenholt, R. W. 1988, Methods Enzymol. 167, 68-92). The E. coli strain J53 containing the plasmid RP4 and the E. coli strain HB101 containing the plasmid pRL623 (gifts from Dr. Wolk) are used for the transfer, of the expression plasmid, by conjugation in Anabaena.

The Anabaena sp. PCC 7120 strain was transformed by conjugation according to the method described by Elhai and Wolk (1988, Methods Enzymol. 167, 747-754) with the expression vectors derived from pNir. The cells transformed with the expression vector pNir were plated out on a BG-11 agar medium containing 10 mM NH₄Cl and 10 mM N-tris(hydroxymethyl)methyl-2-aminoethanesulfonic acid (TES), pH 8. After 24 hours, neomycin was added to the agar in a proportion of 100 μg/ml. The incubation was continued until appearance of the colonies (approximately 8 days). The transformants were then cultured in 100 μl of BG-11 medium containing 15 μg/ml of neomycin, 10 mM NH₄Cl, and 10 mM TES pH 8, diluted 50/50 in a conditioned BG-11 medium containing 10 mM NH₄Cl and 10 mM TES, pH 8. The conditioned medium is a medium that has been used beforehand to culture the wild-type Anabaena sp. PCC 7120 strain, and then sterilized by filtration through a 0.22μ filter before use. The 100 μl of transformant preculture were then used to inoculate 1 ml of BG-11 medium containing 10 mM NH₄Cl and 10 mM TES, pH 8, and 15 μg/ml of neomycin. This 1 ml culture was used to inoculate 5 ml of the same BG-11 medium and then, subsequently, 25 ml.

For the transformations of the Anabaena sp. PCC 7120 strain with the expression vectors derived from the vector pTac, the transformation protocol is similar to that used for the expression vectors pNir, except that the solid medium contains NaNO₃ as nitrogen source at a concentration of 500 mg/l. The nitrogen source is also NaNO₃ in the medium used for the amplification. The liquid BG-11 used contains 500 mg/l of NaNO₃ and 300 μg/ml of neomycin. The other parameters of the amplification protocol are unchanged.

In each case, the expression of the protein of interest is under the control of the tac or nir promoter. These expression vectors contain, in addition to their specific promoter, a ColE1 origin of replication that is functional in E. coli, an origin of replication that is functional in Anabaena sp. PCC 7120 (pDU1), an origin of transfer for conjugation and a neomycin resistance gene.

### EXAMPLE NO. 5

### Comparison of the Constitutive nir and tac Systems

The Anabaena cultures were realized at a temperature of 28° C., shaking at 150 rpm and an illumination of 1500 lux. Anabaena sp. PCC 7120 cells transformed with the vector pTacMBP or pTacGyrB(1-219) were resuspended in 50 ml of BG-11 medium containing 300 μg/ml of neomycin and 500 mg/l of NaNO₃ and cultured to an optical density at 700 nm (OD₇₀₀) of approximately 2. In parallel, Anabaena sp. PCC 7120 cells transformed with the vector pNirMBP or pNirGyrB(1-219) were also induced. To do this, the Anabaena sp. PCC 7120 cells transformed and cultured in a BG-11 medium containing 10 mM NH₄Cl, 10 mM TES, pH 8 and 15 μg/ml of neomycin were washed twice with BG-11 medium containing no nitrogen source. After washing, the cells were resuspended, at an OD₇₀₀ of 0.5, in BG11 medium containing 500 mg/l of NaNO₃ and 15 μg/ml of neomycin.

The cell extracts of these cultures were analyzed by polyacrylamide electrophoresis gel in the presence of sodium dodecyl sulfate (SDS-PAGE) and these various expression levels were evaluated by comigrating, on such a gel, a range of known amount of protein. The results are given in Table 1 below:

| | Vectors | | | |
|---|---|---|---|---|
| | | | pNirGyrB | pTacGyrB |
| | pNirMBP | pTacMBP | (1-219) | (1-219) |
| Proteins | MBP | | GyrB(1-219) | |
| Expression | 250 ± 10 | 125 ± 10 | 100 ± 3 | 15 ± 0.5 |
| level in | | | | |
| mg/l | | | | |

The GyrB(1-219) protein is expressed approximately 10 times less in the constitutive tac system compared with the nir system. In the case of the MBP protein, this ratio is 2. All the trials carried out, moreover, showed that the constitutive tac system does not make it possible to achieve expression levels as high as those obtained with the nir system.

### EXAMPLE NO. 6

### Comparison of the Induced tac and nir Systems

The inducible tac and nir systems were compared using MBP as test protein. Anabaena sp. PCC 7120 cells transformed with the vector pTacIndMBP were induced in the presence of 1 mM IPTG.

The induction was carried out over 7 days, and each day, cells were removed. In parallel, Anabaena sp. PCC 7120 cells transformed with the vector pNirMBP were also induced. In this case, the induction was also carried out over 7 days. Nontransformed Anabaena sp. PCC 7120 cells were cultured, as a negative control for expression, in BG-11 medium containing either 500 mg/l of NaNO₃, or 10 mM NH₄Cl and 10 mM TES, pH 8. FIGS. 1A and 1B show the SDS-PAGE analysis of the cell extracts of aliquots of the induced Anabaena sp. PCC 7120 cells transformed with the vector pNirMBP or pTacIndMBP, as a function of time. The nir system makes it possible to obtain an MBP protein expression level that is approximately 10 times higher from day 4 (FIG. 1A) compared with the tac system (FIG. 1B).

The nir system is therefore a very efficient inducible expression system which allows, in a few days, the accumulation of the order of 250 mg/l of MBP in cyanobacteria.

### EXAMPLE NO. 7

### Regulation of the Expression of the E. coli tac and nir Systems in Anabaena sp. PCC 7120

To study the regulation of the nir and tac promoters, the basal expression levels of MBP were tested in the noninduced cell extracts.

Cell extracts of noninduced cells and of 5-day-induced cells of Anabaena sp. PCC 7120 transformed either with the plasmid pNirMBP or the plasmid pTacIndMBP were analyzed by SDS-PAGE. The proteins were then transferred onto a nitrocellulose membrane and detected using a rabbit anti-MBP polyclonal antibody (New England Biolabs), by chemiluminescence. FIG. 2 shows that, in the absence of inducer, no protein is detected in the cell extract of Anabaena transformed with the vector pNirMBP, whereas the MBP protein is demonstrated in the cell extract of Anabaena transformed with the vector pTacIndMBP. The same observations were made with the GyrB(1-219) protein. Thus, the nir promoter is repressed better in the absence of inducer than the tac promoter.

### EXAMPLE NO. 8

### Expression of Toxic Proteins in Anabaena sp. PCC 7120

The nir system was also tested for the production of the E6 protein and of its C-terminal domain (YZD2), expressed in the form of fusions. When these polypeptides are expressed in E. coli, they are found to be toxic and require the use of a highly regulated expression system (G. Travé, personal communication).

The three fusion proteins GST-E6, MBP-E6 and MBP-YZD2 were tested in the constitutive tac system in Anabaena sp. PCC 7120. In this case, a loss of expression during the growth of the cells was observed, which loss of expression is earlier in the case of the GST-E6 and MBP-E6 proteins, where it appears from the second amplification step. For the MBP-YZD2 protein, the loss of expression was demonstrated when the culture reached a volume of 200 ml. In the two cases, this loss of expression is related to a rearrangement of the plasmid contained in the transformed cells. This was demonstrated by comparing the restriction profile, after digestion with the NdeI, SpeI, XhoI restriction enzymes, of the expression vector of origin before transformation and that of the vector re-extracted from the transformed Anabaena cells no longer expressing the protein. When these proteins were tested in the nir system, no loss of expression was observed.

Table 2 summarizes the mean values of the expression levels obtained with the constitutive tac and nir systems for the GST-E6, MBP-E6 and MBP-YZD2 polypeptides.

| Vectors | Proteins | Expression level in mg/l |
|---|---|---|
| pNirMBP-YZD2 | MBP-YZD2 | 10 ± 1 |
| pTacMBP-YZD2 | | 0 |
| pNirGST-E6 | GST-E6 | 10 ± 1 |
| pTacGST-E6 | | 0 |
| pNirMBP-E6 | MBP-E6 | 12 + 1 |
| pTacMBP-E6 | | 0 |

Expression levels of the order of 10 mg/l were thus obtained after induction. The nir system is therefore sufficiently regulated to allow the production of toxic proteins in Anabaena sp. PCC 7120.

### EXAMPLE NO. 9

### Expression of GFPuv and of β-Galactosidase from E. coli with the nir System in Anabaena sp. PCC 7120

Anabaena sp. PCC 7120 cells transformed with the vector pNir containing the gene for GFPuv were induced for 5 days in a BG-11 medium containing nitrate. The analysis of these induced cells, by fluorescence microscopy, revealed that all the cells analyzed were fluorescent, indicating that GFP was expressed in all the cells homogeneously (see FIG. 3).

This observation was confirmed with another protein: E. coli β-galactosidase. Anabaena sp. PCC 7120 cells transformed with the vector pNir containing the lacZ gene were induced for 3 days in a BG-11 medium containing nitrate. These cells were then fixed with a 4% paraformaldehyde solution, and then incubated for 5 hours in the presence of 5 mM K₃Fe(CN)₆, 5 mM K₄Fe(CN)₆, 1 mg/ml 5-bromo-4-chloro-3-indolyl-β-D-galactopyranoside and 2 mM MgCl₂. The analysis by visible microscopy showed a homogeneous blue staining in all the cells, indicating β-galactosidase expression in all the cells analyzed (see FIG. 3).

The GFP and the β-galactosidase were produced in Anabaena with the nir expression system with a yield of the order of 50 mg/l.

### EXAMPLE NO. 10

### Production of Protein which is Insoluble in E. coli, in Soluble Form with the nir Expression System in Anabaena sp. PCC 7120

MBP expressed under the control of the nir promoter can represent up to 30% of the cell proteins. When such amounts of recombinant proteins are obtained in E. coli, they are generally accumulated in the form of inclusion bodies.

The gene encoding an insoluble mutant of MBP, malE31 (Betton and Hoffnung, 1996, J. Biol. Chem. 271, 8046-8052) was cloned by PCR into the vector pNir. The PCR was carried out in two steps. In a first step, the mutations were introduced. Two overlapping PCR fragments were obtained using the vector pNirMBP as template and the following two pairs of oligonucleotides: oligo 1, 5′-CGCGCGAATTCATGAAAATCGAAGAAGGTA and oligo 2, 5′-GACTTTAGGATCGGTATCTTTCTCGAATTTCTTA; oligo 3, 5′-GATACCGATCCTAAAGTCACCGTTGAGCATCC and oligo 4, 5′-CGCGCGGGATCCCTATGAAATCCTTCCCTCGATCCC. The two PCR fragments were then purified and mixed in an equimolar manner and then reamplified with the oligos 1 and 4, so as to obtain a fragment corresponding to the malE31 gene. This fragment was digested with the EcoRI and BamHI restriction enzymes and inserted into the vector pNir digested beforehand with the same enzymes and dephosphorylated. Anabaena sp. PCC 7120 cells transformed with the vector pNir containing the mutant malE31 were induced for 4 days. The cells were then lyzed by sonication. The cell extracts were centrifuged for 10 min at 10 000 rpm and the supernatant corresponding to the soluble fraction was separated from the pellet (insoluble fraction). The SDS-PAGE analysis of an aliquot of the soluble and insoluble fractions showed that the malE31 polypeptide produced was present essentially in the soluble fraction. This protein, which is insoluble in E. coli and produced in the form of inclusion bodies, can therefore be accumulated in soluble form in Anabaena at an expression level equivalent to that of the wild-type protein.

### EXAMPLE NO. 11

### Production of ¹⁴C-Labeled Proteins

The nir expression system was used to produce the GyrB(1-219) protein labeled with ¹⁴C. Anabaena sp. PCC 7120 cells transformed with the vector pNirGyrB(1-219) were induced for 4 days in a BG-11 medium containing 33 mg/l of NaH¹⁴CO₃ (1 mCi, 52 mCi/mmol, NEN Life Science Products, Zaventem, Belgium), 500 mg/l of NaNO₃ and 15 μg/ml of neomycin. The cells were incubated at a temperature of 28° C. under an illumination of 1500 lux in a hermetically closed culture system. The SDS-PAGE analysis and autoradiography of extracts of Anabaena cells cultured in the presence of NaH¹⁴CO₃ showed that all the cell proteins were uniformly labeled with ¹⁴C. The GyrB(1-219) protein thus labeled was purified according to the method described by (Desplancq et al., 2001, Protein Express. Purif. 23, 201-217). The degree of ¹⁴C incorporation thereof was determined using 20 μl of a solution of protein purified from the induced cell extracts. The counts per minute (cpm) were determined by counting in the presence of 2 ml of scintillation fluid in a radioactivity counter (Packard, Groningen, the Netherlands).

The specific activity of the purified protein was 4×10¹⁴ cpm/mol. The nir system therefore makes it possible to carry out metabolic ¹⁴C-labeling of recombinant proteins.

### EXAMPLE NO. 12

### Production of ¹⁵N, ¹³C, ²H-labeled Proteins

The GyrB(1-219) protein was used with the nir system for the overproduction of ¹⁵N, ¹³C, ²H-labeled recombinant proteins. A preculture of cells is first prepared in 600 ml of BG-11 medium containing 10 mM NH₄Cl and 10 mM TES, pH 8, and 15 μg/ml of neomycin. The cells are incubated for 5 days until an OD₇₀₀=1.5-2 is obtained, at a temperature of 28° C., shaking at 150 rpm and an illumination of 1500 lux, and then centrifuged so as to eliminate the medium containing the NH₄Cl. They are then used to inoculate two liters of BG-11 containing 0.5 g/l of NaNO₃, 0.5 g/l of NaHCO₃ and 15 μg/ml of neomycin in a fermenter. This fermenter is equipped with pH, O₂ and temperature sensors which make it possible to automatically regulate the pH at a value of 8 and the temperature at a value of 28° C. and to monitor the NaHCO₃ consumption, by on-line analysis of the oxygen production of the cells. The culture is realized over a period of 5 days with the daily addition of 1 g/l of NaHCO₃. The shaking in the fermenter is at 100 rpm and the culture is subjected to a constant stream of argon throughout the experiment. The fermenter is illuminated with two light sources, each having an intensity of 4000 lux.

Under these conditions, using only Na¹⁵NO₃ as labeled substrate, the GyrB(1-219) was produced with a uniform enrichment in ¹⁵N of 90%. In a double labeling with ¹⁵N and ¹³C, carried out with the labeled substrates Na¹⁵NO₃ and NaH¹³CO₃, the GyrB(1-219) protein was uniformly enriched in ¹⁵N and ¹³C, with an enrichment of greater than 90% for each of the isotopes. This enrichment is equivalent to that obtained previously with Anabaena sp. PCC 7120 and the expression vector pTac (Desplancq et al., 2001, Protein Express. Purif. 23, 201-217).

Using ²H₂O in place of H₂O in the culture medium, the GyrB(1-219) protein was produced strongly enriched in ²H. For this type of labeling, it is necessary, beforehand, to adapt the transformed Anabaena sp. PCC 7120 cells to growth in a BG-11 medium containing 50% of ²H₂O, then 70% and, finally, 90%. Thus, before transfer into the fermenter, the preculture is prepared in deuterated medium at the desired concentration for the final enrichment. A gradual enrichment in ²H (from 41% to 90%) of the GyrB(1-219) protein was observed for concentrations of 60 to 99.8% of ²H₂O in the culture medium. All the enrichments observed were measured using a mass spectrometer.

In triple labeling experiments carried out with the labeled substrates Na¹⁵NO₃ and NaH¹³CO₃ in a medium containing 92.5% ²H₂O, the NMR analysis of the purified GyrB(1-219) protein showed that it is also possible to enrich in ²H the methyl groups of certain amino acids. In the case of E. coli, this requires the use of deuterated glucose (Bruno Kieffer, personal communication). The labeling method developed in Anabaena sp. PCC 7120 thus makes it possible to simply label, with ²H₂O, methyl groups for which the labeling in E. coli would require the use of complex substrates. Finally, with Anabaena sp. PCC 7120 and the nir expression system, it would appear that the expression of recombinant proteins is not significantly affected when the cells are cultured in highly deuterated media (>90% ²H₂O). On the other hand, in E. coli, a decrease in the level of expression is commonly observed when culturing is carried out in the presence of a high level of ²H₂O (>90%) in the culture medium.

1.-17. (canceled)

18. A method for expressing recombinant proteins comprising:
introducing into cyanobacteria a sequence encoding a protein downstream of an inducible cyanobacterial transcription promoter sequence;
inducing the expression of the protein; and
isolating the recombinant proteins synthesized.

19. The method of claim 18, wherein the transcription promoter sequence comprises a cyanobacterial nir operon.

20. The method of claim 18, wherein the transcription promoter sequence is induced by one or more compounds selected from the group consisting of nitrates or nitrites.

21. The method of claim 18, wherein the transcription promoter sequence is induced by NaNO₃.

22. The method of claim 18, wherein the cyanobacterium is a cyanobacterium Anabaena.

23. The method of claim 22, wherein the cyanobacterium is Anabaena sp. PCC 7120.

24. The method of claim 18, wherein the cyanobacteria is cultured in a medium comprising ¹³C, ¹⁵N, or ²H.

25. The method of claim 18, wherein the cyanobacteria is cultured in a medium comprising Na¹⁵NO₃.

26. The method of claim 18, wherein the expressed recombinant protein is toxic for the cyanobacteria.

27. A vector comprising a DNA sequence encoding a recombinant protein under the control of an inducible cyanobacterial transcription promoter sequence.

28. The vector of claim 27, wherein the inducible cyanobacterial transcription promoter sequence comprises a cyanobacterial nir operon.

29. A method of expressing recombinant proteins comprising:
introducing into cyanobacteria the vector of claim 27;
inducing the expression of the protein; and
isolating the recombinant proteins synthesized.

30. The method of claim 29, wherein the cyanobacteria is cultured in a medium comprising ¹³C, ¹⁵N, or ²H.

31. The method of claim 29, wherein the cyanobacteria is cultured in a medium comprising Na¹⁵NO₃.

32. A cyanobacterium transformed with the vector of claim 27.

33. The cyanobacterium of claim 32, wherein the cyanobacterium is a cyanobacterium Anabaena.

34. The cyanobacterium of claim 33, wherein the cyanobacterium is Anabaena sp. PCC 7120.


SEQUENCE LISTING (10 sequences)

SEQ ID NO: 1 - DNA, 25 bp, Artificial Sequence
  Synthetic primer
ggccgcaggcctctcgagcccgggg

SEQ ID NO: 2 - DNA, 25 bp, Artificial Sequence
  Synthetic primer
gatcccccgggctcgagaggcctgc

SEQ ID NO: 3 - DNA, 39 bp, Artificial Sequence
  Synthetic primer
atccggggtctcccatgtttcaggacccacaggagcgac

SEQ ID NO: 4 - DNA, 52 bp, Artificial Sequence
  Synthetic primer
atccggggtctcggtaccgcggccgcttacagctgggtttctctacgtgttc

SEQ ID NO: 5 - DNA, 37 bp, Artificial Sequence
  Synthetic primer
gcgcgcagatctagctactcattagttaagtgtaatg

SEQ ID NO: 6 - DNA, 44 bp, Artificial Sequence
  Synthetic primer
ggccggggatccgaattcgttctcataaagtttttttgctcaag

SEQ ID NO: 7 - DNA, 30 bp, Artificial Sequence
  Synthetic primer
cgcgcgaattcatgaaaatcgaagaaggta

SEQ ID NO: 8 - DNA, 34 bp, Artificial Sequence
  Synthetic primer
gactttaggatcggtatctttctcgaatttctta

SEQ ID NO: 9 - DNA, 32 bp, Artificial Sequence
  Synthetic primer
gataccgatcctaaagtcaccgttgagcatcc

SEQ ID NO: 10 - DNA, 36 bp, Artificial Sequence
  Synthetic primer
cgcgcgggatccctatgaaatccttccctcgatccc